(19) United States
(12) Patent Application Publication
(10) Pub. No.: US 2014/0286595 A1
(43) Pub. Date: Sep. 25, 2014

(54) Safe and easy bagging tool a tool for safe and easy bagging of instruments to be sterilized

(71) Applicant: Richard Charl Moreschini, Pueblo, CO (US)

(72) Inventor: Richard Charl Moreschini, Pueblo, CO (US)

(21) Appl. No.: 13/986,025

(22) Filed: Mar. 25, 2013

(51) Int. Cl. A61B 19/02

(52) U.S. Cl. CPC A61B 19/026; A61B 2019/0267
USPC 383/33

(57) ABSTRACT

This invention, “Safe and Easy” bagging tool, is a very simple tool, made of flexible metal or plastic, about seven inches long, shaped somewhat like a flat funnel, that will hold a baggie open, (a self sealing envelope shaped paper and plastic bag used to hold and store surgical instruments to be sterilized.) so it will be easier to put the instruments into the baggie, and at the same time, it is acting as barrier between the baggie and the operators hand that is holding said baggie, preventing the chance of an exposure, from an accidental puncture or cut to the operators hand, from a sharp or pointed, instrument, being loaded into the bag.

### BACKGROUND OF THE INVENTION

This invention relates to devices used in medical facilities in general and sterilization of instruments specifically.

As part of the medical professions, instruments are used, they become contaminated, and must be cleaned, sealed in a paper like baggie, and then be sterilized, and stored in a drawer or cabinet, till they are needed again.

A common problem with the known devices of conventional type, and use, is the difficulty in placing the used and cleaned instruments into the self seal-able baggies so that they can be sterilized, and then stored, until needed again.

“Baggies,” are a sterilization pouch, a self sealing, open-ended envelope type bag that is clear plastic on one side and a paper material on the other side. They are placed in a sterilizer (steam autoclave-chemclave or dry heat) with the paper side up, to allow moisture to escape. Instruments can be stored in these bags indefinitely and be considered “sterile” as long as there are no rips, tares, or holes poked in them. It is hard to hold the bag open and put instruments in carefully with out a cut, scratch or exposure. Frequently. while placing the instruments into the bag, a sharp instrument will tear through the paper side, requiring a new bag, plus the very serious threat to the operator of being exposed to what ever micro organism the instrument might have on it.

### BRIEF SUMMARY OF THE INVENTION

The present invention is sort of a narrow, funnel like' shaped piece of flexible metal or plastic about seven inches long and about five inches wide. This invention is very simple and is used, very much like a funnel. is used; (making it easier to put something inside of something else,) It can be used by slipping it over the baggie, or by slipping it inside the baggie, making it easier to place the contaminated surgical tools into the baggies, and at the same time, it is placing a shield between the contaminated instruments and the hand of the operator who is holding the baggie. This invention is used with an item needed in the sterilization of surgical instruments, called a “baggie.” This baggie is a self sealing, open ended envelope type bag that is clear plastic on one side, and has a thin paper like material on the back, or bottom side .The used instruments are cleaned of debris then placed into the paper baggies, sealed, then placed in a sterilizer, (autoclave,-chemclave or dry heat,) then sterilized. After sterilization, the baggie and its contents can be stored indefinitely and be considered “sterile” as long as there are no rips, tares or holes, in the baggie.

### OBJECTS OF THE INVENTION

An object of the present invention is to make the procedure of bagging used Surgical instruments easier.

Another object of the present invention is to make the procedure of bagging used surgical instruments less hazardous to the operator.

Another object of the present invention is to make the procedure of bagging used surgical instruments less costly.

These and other objects and advantages of this invention will become apparent upon reading the following descriptions of which the attached drawings form a part.

### BRIEF DESCRIPTION OF THE DRAWINGS

FIG. 1 Is a perspective view of the flexible metal or plastic “Safe and Easy” bagging Tool of the present invention.

FIG. 2. Is a perspective view of the flexible, metal or plastic “Safe and Easy” bagging Tool, of the present invention, with an opened baggie slipped into the bagging Tool.

FIG. 3. Is a perspective view of the flexible, metal or plastic “Safe and Easy” bagging tool, of the present invention, that has been slipped into an open baggie.

### REFERRING TO THE PRESENT INVENTION

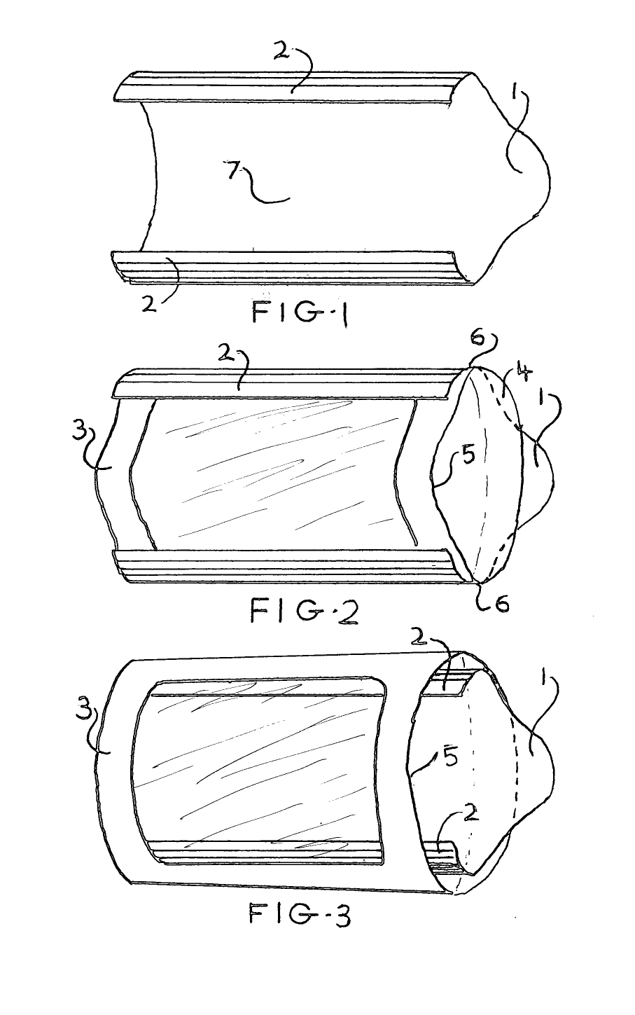

In FIG. 1 We see a flexible metal or plastic “Safe and Easy” bagging tool 1, With the bent upwards sides 2, and the solid unbroken back portion 7, of the tool 1, that acts as the shield, or barrier preventing any sharp or pointed parts of the instruments from accidentally injuring the hand of the operator.

In FIG. 2. We see a flexible metal or plastic “Safe and Easy” bagging tool 1, with a Baggie 3, in it, and held open 5, by the bent over ends 2, of bagging tool 1, pressing against the sides 6, of the baggie 3, now ready to be safely loaded with instruments. After the instruments are loaded, the baggie top 4. is closed sealing the baggie 3, ready to be removed from the bagging tool 1, and ready to be placed in a sterilizer.

In FIG. 3. We see a flexible metal or plastic “Safe and Easy” bagging tool 1, inserted Into an open baggie 3. and the baggie 3, being held open 5, by the upwards bent sides 2, of the bagging tool 1, making it ready to be loaded with the unsterilized surgical instruments.

This invention may be further developed within the scope of the following attached claims, accordingly, it is desired that the fore going description be read merely as being illustrative of an operative embodiment of this invention and not a strictly limiting sense.

What is claimed is:

1. A “Safe and Easy” bagging tool that will hold a sterilization baggie open, making it easier for an operator to insert used surgical instruments into it, and at the same time, automatically act as a barrier between the baggie with its contents, from the hand of the operator who is holding it, preventing the chances, of an accidental puncture or cut from a sharp or pointed, and contaminated, instrument being handled or loaded into the baggie.

2. A claim as set down in claim 1, wherein the Safe and Easy bagging tool is on the outside of the baggie, and the shield or barrier is located outside and beneath the lower surface of the bottom material of the baggie.

3. A claim as set down in claim 1, wherein the Safe and Easy bagging tool in on the inside of the baggie, and the shield or barrier is located inside and between the used instruments and the upper surface of the lower sheet of material of the baggie.
